(19) United States
(12) Patent Application Publication
(10) Pub. No.: US 2026/0000324 A1
(43) Pub. Date: Jan. 1, 2026

(54) ADHESIVE PAD WITH A METALLIC COIL FOR SECURING AN ON-BODY MEDICAL DEVICE

(71) Applicant: INSULET CORPORATION, Acton, MA (US)

(72) Inventors: David NAZZARO, Groveland, MA (US); John D'ARCO, Wilmington, MA (US); Nicholas CONTE, Harvard, MA (US); Kepei SUN, Andover, MA (US)

(21) Appl. No.: 19/320,286

(22) Filed: Sep. 5, 2025

Related U.S. Application Data
(63) Continuation of application No. 17/554,455, filed on Dec. 17, 2021, now Pat. No. 12,433,512.
(60) Provisional application No. 63/127,328, filed on Dec. 18, 2020.

(51) Int. Cl. A61B 5/145; A61B 5/00; A61B 5/1486

(52) U.S. Cl. CPC A61B 5/14532; A61B 5/6833; A61B 5/14865; A61M 2205/3569; A61M 2230/201

(57) ABSTRACT

Exemplary embodiments may provide an adhesive pad that is designed to be used with an on-body medical device. The adhesive pad may partially or fully surround a perimeter of the on- body medical device. The adhesive pad may be the primary mechanism or may provide an additional mechanism for helping to secure the on-body medical device to the user. In addition, the adhesive pad may include a metallic coil, such as a metal loop, that is woven into the adhesive pad or otherwise inserted into or secured to the adhesive pad. The metallic coil may play multiple roles. First, the metallic coil may act as an antenna to facilitate wireless communications with the on-body medical device, such as NFC communications. In addition, the metallic coil may serve as a power source for electric components positioned on the adhesive pad.

### RELATED APPLICATIONS

This application is a continuation of U.S. Patent Application No. 17/554,455, filed December 17, 2021, which claims the benefit of U.S. Provisional Patent Application No. 63/127,328, filed December 18, 2020, the contents of which are incorporated herein by reference in their entirety.

### BACKGROUND

On-body medical devices need to be secured to a user. One way to secure to an on- body medical device is to provide an adhesive. The adhesive may be applied directly to a bottom surface of the on-body drug delivery device and then adhered to skin of the user. Alternatively, an adhesive pad may be secured to the on-body medical device and an adhesive-covered side of the adhesive pad may be stuck to skin of the user.

On-body medical devices have little room for additional electronic components. Typically, electronic components are situated on a printed circuit board that has no free space for additional components. There is a general desire to keep on-body medical devices small in size so as to be less cumbersome to a user and to be more readily situated out of sight, such as by being situated under clothing or in areas of the body like the back of the arm that are not usually as visible to an observer.

### SUMMARY

In accordance with an inventive aspect, an adhesive pad is for use with an on-body medical device. The adhesive pad has a geometry configured to at least partially surround the on-body medical device. The adhesive pad includes an adhesive on at least one side of the adhesive pad for securing the adhesive pad to skin of a user. The novel adhesive pad described herein also includes a metallic coil for acting as an antenna for wireless communications to and from the on-body medical device. The novel adhesive pad may provide physiological sensing mechanisms or arrays. The novel adhesive pad may have components with the ability to process data, transmit information to other devices or act as a hub or conduit for multiple on-body devices.

The adhesive pad may include a power source secured or electrically connected to the adhesive pad for providing power. The power source may be, for example, one of a battery power source or an energy harvesting device. The adhesive pad may include an electronic component electrically coupled to the metallic coil. The electronic component may be a sensor for sensing a biometric of the user. The sensor may measure at least one of activity, heart rate, moisture, temperature, an analyte level (such as glucose level), or sound pressure. At least one additional sensor may also be electrically coupled to the metallic coil. The electronic component may be a system on a chip (SoC) or a wireless communications component. The metallic coil may serve as an inductive coil for powering the electronic component.

In accordance with an inventive aspect, a system may include an on-body medical device and an adhesive pad for use with an on-body medical device. The adhesive pad may have a geometry configured to at least partially surround the on-body medical device. The adhesive pad may have an adhesive on at least one side of the adhesive pad for securing the adhesive pad to skin of a user. The adhesive pad may include a metallic coil for acting as an antenna for wireless communications to and from the on-body medical device.

The on-body medical device may be a drug pump such as an insulin pump. The on- body medical device may include an inductive coil. The metallic coil of the adhesive pad and the inductive coil of the on-body medical device may communicate via Near Field Communication (NFC). The metallic coil and the inductive coil may be configured to create magnetic fields to induce current in each other. An electronic component may be electrically connected to the metallic coil. The adhesive pad may be secured to a skin of the user and may overlap with a portion of the on-body medical device to help secure the on-body medical device to the user.

In accordance with an inventive aspect, a method comprises including a metallic coil in an on-body medical device and including a metallic coil in an adhesive pad in proximity to the on-body medical device. The method further comprises configuring the metallic coil in the adhesive pad as an antenna for wireless communications. The method may further include securing an electronic component to the adhesive pad and electronically connecting the electronic component to the metallic coil to provide power to the electronic component. The on- body medical device may be a drug pump such as an insulin pump.

### BRIEF DESCRIPTION OF THE DRAWINGS

FIG. 1 depicts an illustrative drug delivery system for an exemplary embodiment that includes an on-body drug delivery device and an adhesive pad.

FIG. 2A depicts an illustrative user wearing an on-body drug delivery device and adhesive pad of an exemplary embodiment.

FIG. 2B depicts illustrative alternative geometries for an adhesive pad of an exemplary embodiment.

FIG. 3 depicts an illustrative arrangement of components secured to an adhesive pad of an exemplary embodiment.

FIG. 4 depicts an adhesive pad and an on-body drug delivery device positioned relative to each other when worn by a user in accordance with an exemplary embodiment.

FIG. 5 depicts a flowchart of illustrative steps that may be performed to use metallic coils of the on-body drug delivery device and adhesive pad in an exemplary embodiment.

FIG. 6A depicts a diagram of different types of power sources that may be used with an adhesive pad in an exemplary embodiment.

FIG. 6B depicts a diagram of different types of sensors that may be secured to the adhesive pad in accordance with an exemplary embodiment.

FIG. 7 depicts a flowchart showing illustrative steps that may be performed in an exemplary embodiment to use data from sensors.

FIG. 8A depicts an arrangement in which a metallic coil of an adhesive pad couples with a metallic coil of an on-body drug delivery device via a bottom cover of the on-body drug delivery device in an exemplary embodiment.

FIG. 8B depicts an arrangement in which a metallic coil of an adhesive pad couples with a metallic coil of an on-body drug delivery device via a top cover of the on-body drug delivery device in an exemplary embodiment.

FIG. 9A depicts an illustrative arrangement where electronic components of the on- body drug delivery device are electrically connected via wires to an electronic component secured to the adhesive pad in an exemplary embodiment.

FIG. 9B depicts an illustrative arrangement where electronic components of the on- body drug delivery device are electrically connected via wires to an electronic component secured to the adhesive pad and a power source secured to the adhesive pad is electrically connected to an electronic component of the on-body drug delivery device via a wire in an exemplary embodiment.

### DETAILED DESCRIPTION

Exemplary embodiments may provide an adhesive pad that is designed to be used with an on-body medical device. The adhesive pad may partially or fully surround a surface of the on-body medical device. The adhesive pad may provide a primary or an additional mechanism for helping to secure the on-body medical device to the user. For example, a top surface of the adhesive pad may be secured to the on-body medical device via an adhesive or via spot welding, for example, and a bottom surface of the adhesive pad may secure the on-body medical device to the skin of a user. In another example, the on-body medical device may be secured to the user via adhesive placed on a bottom surface of the on-body medical device or by a second adhesive pad that is part of the on-body medical device. The adhesive pad may overlap with the second adhesive pad to be secured to the second adhesive pad and may include additional area that surrounds the on-body medical device that adheres to the skin of the user. The adhesive pad may provide additional surface area that adheres to the skin of the user and that is positioned over at least a portion of the second adhesive pad. As a result, the on-body medical device is held more securely to the user in this example.

In addition, the adhesive pad may include a metallic coil, such as a metal loop, that is woven into the adhesive pad or otherwise inserted into or secured to the adhesive pad. The metallic coil may play multiple roles. First, the metallic coil may act as an antenna to facilitate wireless communications with the on-body medical device, such as NFC communications. In addition, the metallic coil may serve as a power source for electric components positioned on the adhesive pad. The metallic coil may carry current produced by induction due to magnetic fields produced by a corresponding magnetic coil in the on-body medical device. Alternatively, a power source such as a battery-based power source or energy harvesting device may be positioned on the adhesive pad to provide power to the metallic coil. In some exemplary embodiments, power is provided by the on-body medical device.

Electronic components may be positioned on or within the adhesive pad and may be electrically connected to the metallic coil. The metallic coil may provide power to the electronic components. Examples of electronic components that may be positioned on or within the adhesive pad include but are not limited to sensors, a SoC or a wireless communications component for transmitting and receiving wireless communications via the metallic coil.

Thus, the adhesive pad may have the additional advantage of providing additional electronic components, like sensors or an SoC, in proximity to the on-body medical device without occupying valuable space inside the on-body medical device. These electronic components can improve the operation of the on-body medical device without using valuable surface area on the printed circuit board of the on-body medical device.

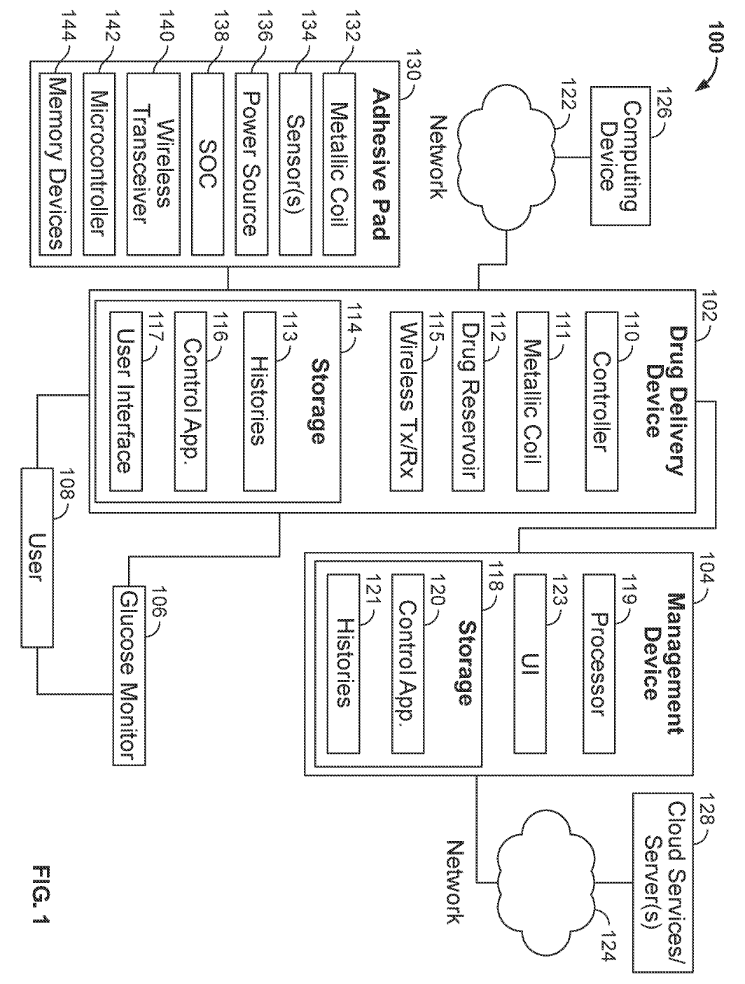

The on-body medical device may be a drug delivery device in some exemplary embodiments. FIG. 1 depicts an illustrative drug delivery system (100) that includes an on- body drug delivery device (102) as the on-body medical device and an adhesive pad (130) that is used with the on-body drug delivery device (102). The on-body drug delivery device (102) may be directly coupled to a user (e.g., directly attached to a body part and/or skin of the user (108) via an adhesive or the like). In an example, a surface of the on-body drug delivery device (102) may include an adhesive to facilitate attachment to the user (108).

The on-body drug delivery device (102) may include a controller (110). The controller (110) may be implemented in hardware, software, or any combination thereof. The controller (110) may, for example, be a microprocessor, a logic circuit, a field programmable gate array (FPGA), an application specific integrated circuit (ASIC) or a microcontroller coupled to a memory. The controller (110) may maintain a date and time as well as other functions (e.g., calculations or the like). The controller (110) may be operable to execute a control application (116) stored in the storage (114) that enables the controller (110) to direct operation of the on- body drug delivery device (102). The storage (114) may hold histories (113) for a user. Where the on-body drug delivery device (102) is an insulin delivery device, the histories (113) may include information such as a history of automated insulin deliveries, a history of bolus insulin deliveries, meal event history, exercise event history, and the like. In addition, the controller (110) may be operable to receive data or information. The storage (114) may include both primary memory and secondary memory. The storage (114) may include random access memory (RAM), read only memory (ROM), optical storage, magnetic storage, removable storage media, solid state storage or the like.

The on-body drug delivery device (102) may include a drug reservoir (112) for storing a drug, such as insulin, for delivery to the user (108) as warranted. A fluid path to the user (108) may be provided, and the on-body drug delivery device (102) may expel the drug from the drug reservoir (112) to deliver the drug to the user (108) via the fluid path. The fluid path may, for example, include tubing coupling the on-body drug delivery device (102) to the user (108) (e.g., tubing coupling a cannula to the drug reservoir (112)).

There may be one or more communications links with one or more devices physically separated from the on-body drug delivery device (102) including, for example, a management device (104) of the user and/or a caregiver of the user, a glucose monitor (106) and/or an adhesive pad (130). The drug delivery device (102) may include a metallic coil (111) that acts as an antenna. In addition, a wireless transceiver (115) may be provided to transmit and receive wireless communications. The communication links may include any wired or wireless communication link operating according to any known communications protocol or standard, such as the Bluetooth® standard, the Bluetooth® Low Energy (BLE) standard, Wi-Fi (IEEE 802.11), the NFC standard, a cellular standard, the Wireless Body Area Network Standard (see IEEE 802.15.06) or any other wireless protocol or standard. The on-body drug delivery device (102) may also include a user interface (117), such as an integrated display device for displaying information to the user (108) and in some embodiments, receiving information from the user (108). The user interface (117) may include a touchscreen and/or one or more input devices, such as buttons, knob or a keyboard.

The on-body drug delivery device (102) may interface with a network (122). The network (122) may include a local area network (LAN), a wide area network (WAN) or a combination therein. A computing device (126) may be interfaced with the network, and the computing device may communicate with the on-body drug delivery device (102).

The drug delivery system (100) may include a glucose monitor (106) for sensing the blood glucose concentration levels of the user (108). The glucose monitor (106) may provide periodic blood glucose concentration measurements and may be a continuous glucose monitor (CGM), or another type of device or sensor that provides blood glucose measurements. The glucose monitor (106) may be physically separate from the on-body drug delivery device (102) or may be an integrated component thereof. The glucose monitor (106) may provide the controller (110) with data indicative of measured or detected blood glucose levels of the user (108). The glucose monitor (106) may be coupled to the user (108) by, for example, adhesive or the like and may provide information or data on one or more medical conditions and/or physical attributes of the user (108). The information or data provided by the glucose monitor (106) may be used to adjust drug delivery operations of the on-body drug delivery device (102).

The drug delivery system (100) may also include management device (104). The management device (104) may be a special purpose device, such as a dedicated personal diabetes manager (PDM) device. Alternatively, the management device (104) may be a programmed general-purpose device, such as any portable electronic device including, for example, a dedicated controller, such as processor, a smartphone, or a tablet. The management device (104) may be used to activate or program or adjust operation of the on-body drug delivery device (102) and/or the glucose monitor (106). The management device (104) may be any portable electronic device including, for example, a dedicated controller, a smartphone, or a tablet. In the depicted example, the management device (104) may include a processor (119) and a storage (118). The processor (119) may execute processes to manage a user's blood glucose levels and for controlling the delivery of the drug or therapeutic agent to the user (108). The processor (119) may also be operable to execute programming code stored in the storage (118). For example, the storage may be operable to store one or more control applications (120) for execution by the processor (119). The storage (118) may store the control application (120), histories (121) like those described above for the insulin delivery device (102) and other data and/or programs. In another example, after activation, the on-body drug delivery device (102) may operate without the management device (104) by communicating with the glucose monitor (106) and delivering a drug based on those communications using an automated drug delivery (ADD) application or control application (116) stored in memory on the on-body drug delivery device (102).

The management device (104) may include a user interface (123) for communicating with the user (108). The user interface may include a display, such as a touchscreen, for displaying information. The touchscreen may also be used to receive input when it is a touch screen. The user interface (123) may also include input elements, such as a keyboard, button, knobs or the like.

The management device 104 may interface with a network (124), such as a LAN or WAN or combination of such networks. The management device (104) may communicate over network (124) with one or more servers or cloud services (128).

An adhesive pad (130) may be provided in the drug delivery system (100). As was mentioned above, the adhesive pad (130) secures or helps to further secure the on-body drug delivery device to the user (108). In addition, the adhesive pad (130) may contain additional electronic components so that those components need not be incorporated inside the on-body drug delivery device (102), in which space is at a premium. The adhesive pad (130) may include a metallic coil (132), such as a metal loop or a rectangular metal coil, such as found with NFC antennas. The metallic coil (132) may act as an antenna for wireless communications, a conduit for delivering power to electronic components and/or an inductive power source, as will be explained in more detail below.

The adhesive pad (130) may include electronic component(s) that are secured to the adhesive pad (130), such as by being woven into the pad, inserted to or within the pad, or stuck to the pad by adhesive. A first example of such electronic components that are secured to the adhesive pad (130) are sensors (134) for sensing biometric information of the user (108). The electronic components secured to the adhesive pad (130) may also include a power source (136), such as a battery-based power source or an energy harvesting device. The electronic components secured to the adhesive pad (130) may include a System on a Chip (SoC) (138) that may provide computing capability or other functionality.

The electronic components secured to the adhesive pad (130) may include a wireless transceiver or other wireless communications chip (140) enabling wireless communications via the metallic coil (132). For example, the wireless transceiver or other wireless communications chip (140) may enable wireless communications via the NFC standard, the Bluetooth® standard, the Bluetooth® Low Energy (BLE) standard, Wi-Fi (IEEE 802.11), a cellular standard or the Wireless Body Area Network Standard (see IEEE 802.15.06). The adhesive pad (130) may include a microcontroller (142) for processing data and performing operations. Still further, the adhesive pad (130) may include one or more memory devices (144) for storing data and/or instructions. Given the wireless transceiver (140), the microcontroller (142) and the one or more memory devices (144), the adhesive pad (130) may act as a hub or conduit among other on-body medical devices and the drug delivery system (100).

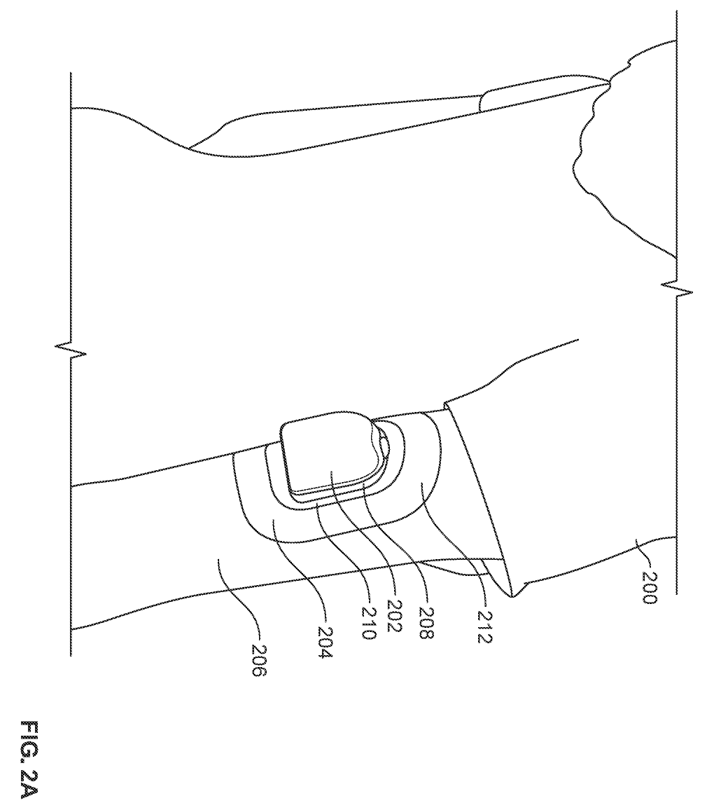

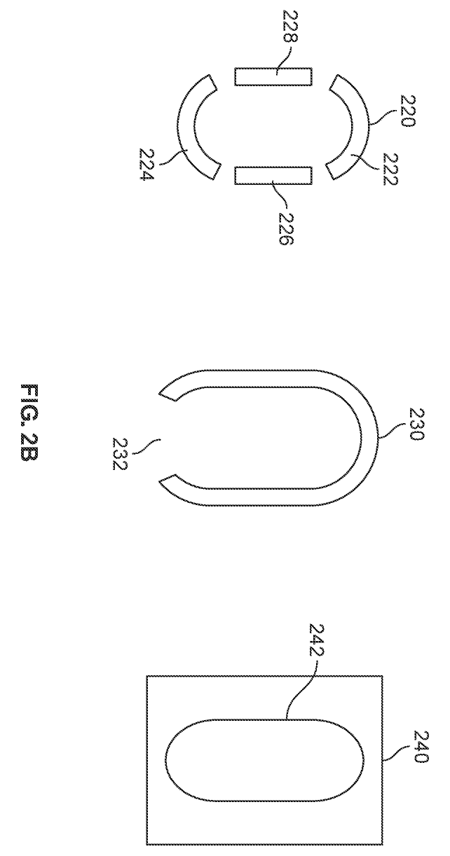

FIG. 2A shows an example of a user (200) wearing an on-body drug delivery device (202) with the adhesive pad (204) fully surrounding the on-body drug delivery device (202). Both the on-body drug delivery device (202) and the adhesive pad (204) are secured to the back of the arm (206) of the user (200). The on-body drug delivery device (202) and the adhesive pad (204) may be secured to other areas of the user (200), such as, for example, the user's abdomen, lower back or leg. In the example shown in FIG. 2A, the on-body drug delivery device (202) includes an adhesive pad (208) secured to a bottom surface of the on-body drug delivery device (202) and to the skin of the user (200) via the adhesive on the adhesive pad (208). The adhesive pad (204) overlaps with the adhesive pad (208) of the on-body drug delivery device (202) in an annular region 210 in the example shown in FIG. 2A. The adhesive on the adhesive pad (204) adheres to the top surface of the adhesive pad (208) of the on-body drug delivery device (202). The non-overlapping region (212) of the adhesive pad (204) adheres to the skin of the patient. As a result, the adhesive pad (204) more greatly secures the on-body drug delivery device (202) to the user (200) versus the conventional use of only the adhesive pad (208) of the on-body medical device (202) to secure the on-body drug delivery device (202) to the user (200).

In an alternative embodiment, the adhesive pad (208) and adhesive pad (204) may be integral, or in other words, one adhesive pad (130) that both (1) houses (or secures therein) additional electronic components not located within the on-body medical device, and (2) secures the on-body medical device to the skin of the user. The additional electronic components may be located in annular region (210), and/or may be located directly under the on-body medical device (102/202), and/or may be located outside of annular region (210).

It will be appreciated that the adhesive pad (204) may assume different geometries than those shown in FIG. 2A. In some geometries, the adhesive pad (204) only partially surrounds the on-body drug delivery device. FIG. 2B shows an example of some alternative geometries for the adhesive pad (204). For instance, in the geometry (220), there are four disjoint segments, including two curved segments (222) and (224) and two straight sections (226) and (228) for positioning around the on-body drug delivery device (202) to partially enclose (in at least one plane) the on-body drug delivery device (202). In some exemplary embodiments, only the straight sections (226) and (228) are used to secure the on-body drug delivery device (202). In other exemplary embodiments, only the curved sections (222) and (224) are used to secure the on-body drug delivery device (202). In geometry (230), the adhesive pad is horseshoe shaped with an opening (232). In geometry (240), the adhesive pad has a geometry akin to that shown in FIG. 2A where the adhesive pad (204) fully surrounds the on- body drug delivery device (202), but the adhesive pad has a rectangular shape rather than an oval shape. It will be appreciated that the geometries of FIGS. 2A and 2B are intended to be illustrative and not exhaustive or limiting.

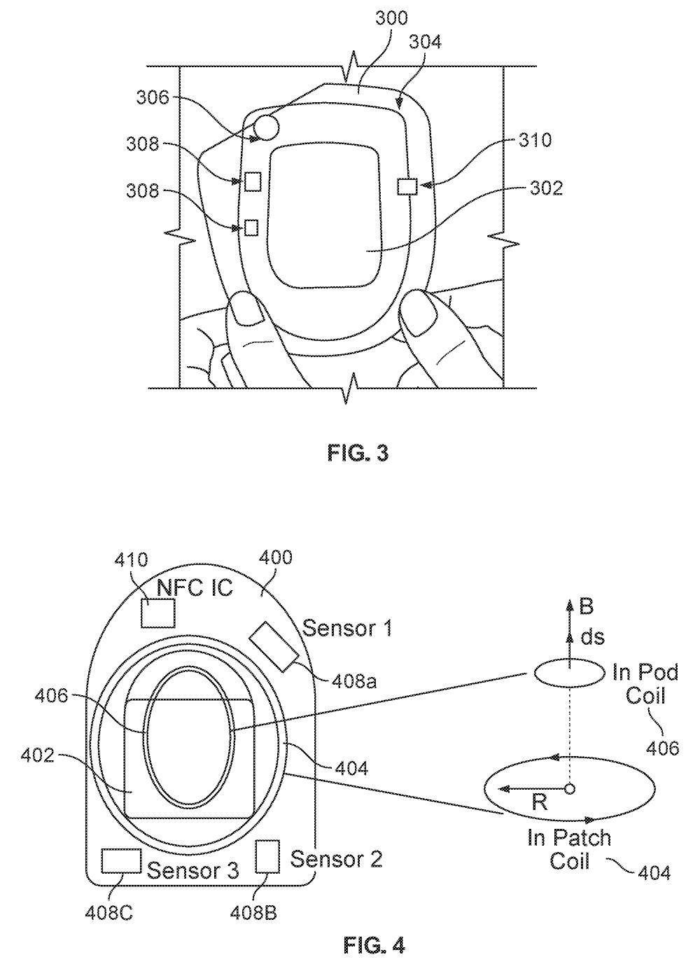

FIG. 3 shows an example of an adhesive pad (300) with a metallic coil (304) embedded or otherwise secured to the adhesive pad (300). As was mentioned above, the metallic coil (304) may be woven, adhered or otherwise secured to the adhesive pad (300). In the example of FIG. 3, the metallic coil (304) has a shape like that of the outline of the on-body drug delivery device (202) (FIG. 2A) from a plan view. In this example, the metallic coil (304) is a simple loop but may assume other forms such as a rectangular arrangement, an arrangement with concentric loops of metal, a zig-zag pattern arrangement or other forms.

The adhesive pad (300) may include a number of electronic components that are electrically connected to the metallic coil (304). For example, a power source (306) may be secured to the adhesive pad (300) and electrically connected to the metallic coil (304). Electronic components like sensors (308) may also be secured to the adhesive pad (300) via, for example, weaving, insertion or adhesive and electrically connected to the metallic coil (304). An additional electronic component (310) may be secured to the adhesive pad (300) and electrically connected to the metallic coil (304). The additional electronic component (310) may be, for example, an SoC that provides computing power or another functionality. Moreover, the additional electronic component (310) may be a wireless communications chip for facilitating wireless communications with the on-body drug delivery device, such as a wireless transceiver and/or related components. The adhesive pad (300) may include multiple layers of material, and the electronic components (306), (308) and (310), in some embodiments, may be positioned between the layers of material. The adhesive pad (300) may include an opening (302) for accommodating the on-body drug delivery device. Alternatively, this region (302) may be where the adhesive pad (300) is secured to the on-body drug delivery device by, for example, spot welding.

FIG. 4 depicts the adhesive pad (400) and the on-body drug delivery device (402) positioned together as when adhered to a user. In the example of FIG. 4, the adhesive pad (400) has a metallic coil ("in-patch coil") (404) secured or positioned within the adhesive pad (400) and sensors (408A, 408B and 408C) secured to or placed within the adhesive pad (400). An integrated circuit (IC) for facilitating NFC communications (410) also is secured to the adhesive pad (400). The on-body drug delivery device (402) also includes a metallic coil ("in- pad coil") (406) positioned within or on top of the on-body drug delivery device (402).

Applying power to one of the metallic coils (404) and (406) causes magnetic fields to be generated. The magnetic fields cause a current to be produced in the other of the metallic coils (404) and (406). The magnetic coils (404) and (406) act as antennas for transmitting and receiving wireless signals, such as NFC signals. The NFC IC (410) may receive from the metallic coil (404) acting as an antenna and may generate and transmit signals with the metallic coil (404) acting as an antenna. The wireless signals may, for example, be sent to the on-body drug delivery device (402) or received from the on-body drug delivery device (402).

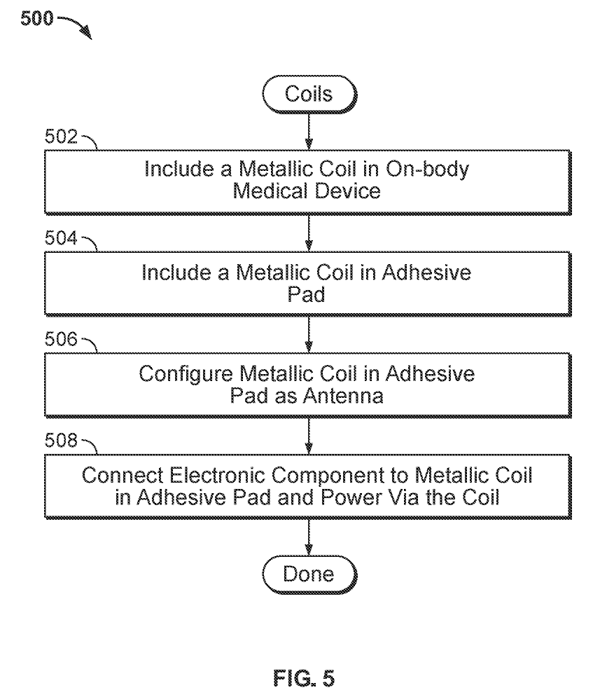

FIG. 5 depicts a flowchart 500 of steps that may be performed in an exemplary embodiment to use the metallic coils, like (404) and (406). First, a metallic coil (406) is included in the on-body drug delivery device (502). A metallic coil (404) also is included in the adhesive pad (400) (504). The metallic coil (404) in the adhesive pad (400) is configured to act as an antenna (506). This may entail connecting a wireless communication IC like NFC IC (410) in FIG. 4 to the metallic coil (404). An electronic component (like (408A), (408B) or (408C)) is connected to the metallic coil (404) on the adhesive pad (400). The metallic coil (404) provides power to the connected electronic component(s) (508). Thus, the metallic coil (404) is not only used as an antenna for wireless communications but also as a power conduit for delivering power to the connected electronic components.

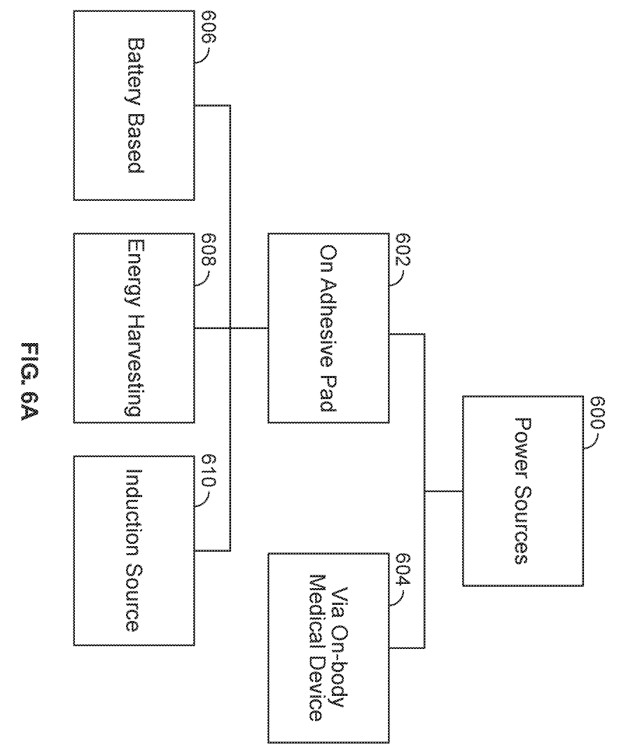

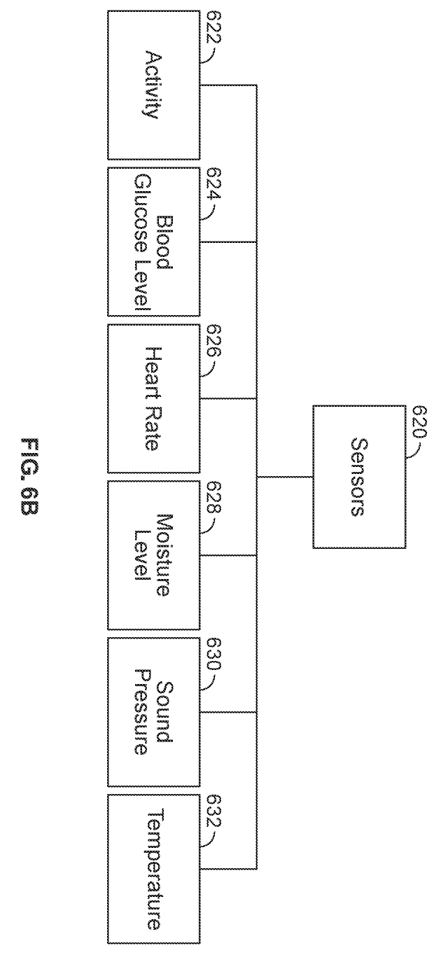

As was discussed above, a power source may be provided on the adhesive pad (400). FIG. 6A depicts a number of different types of power sources (600) that may be used. The power sources may be resident on the adhesive pad (602) or may be provided via the on-body drug delivery device (604). This may entail a wired connection to the power source of the on- body drug delivery device (402), such as a wired connection between the adhesive pad of the on- body drug delivery device (402) and the adhesive pad (400). When the power source is resident on the adhesive pad (400) (602), the power source may be battery-based (606), such as one or more batteries, like low profile button cell batteries. Alternatively, the power source may be an energy harvesting device (608). Examples of energy harvesting devices (608) include solar devices, motion driven energy harvesting devices, thermal energy harvesting devices and the like. Lastly, the energy source may be an induction source (610), such as induction in the metallic coil (404) brought about by magnetic fields from the metallic coil (406) of the drug delivery device (402) when power is applied to the magnetic coil (406).

As has been discussed above, sensors (620) (FIG. 6B) may be secured to the adhesive pad (400). These sensors (620) may take many different forms. A sensor may be an activity sensor (622), like an accelerometer, which measures movement. A sensor may be a sensor of a blood glucose level (624) of the user. A sensor may measure the heart rate (626) of the user. A sensor may measure the moisture level (628), such as on a user's skin, to tell if the user is sweating or not. A sensor may measure sound pressure (630). A sensor may measure the temperature (632) of the user. All these biometric values measured by the sensor(s) (620) may be useful in gauging a current state of the user and may be used by the controller 110 (FIG. 1) in controlling drug delivery to the user.

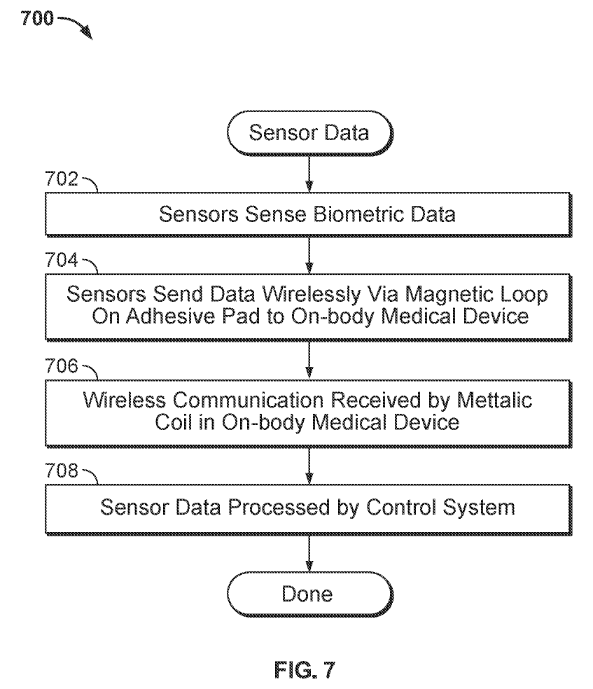

FIG. 7 depicts a flowchart (700) of steps that may be performed in exemplary embodiments with respect to data gathered by sensors, like those types depicted in FIG. 6B. Initially, the sensors sense biometric data (702). For instance, a first sensor may sense the heart rate of the user, and a second sensor may sense the temperature of the user. The sensors send the data wirelessly via the metallic coil (404) on the adhesive pad (400) to the metallic coil (406) of the on-body drug delivery device (402) (704). This may be done at periodic sampling times or when requested. The metallic coil (406) in the on-body drug delivery device (402) receives the sensor data (706). The received sensor data is passed on to the controller (110) and processed in accordance with the control application (116) run on the controller (110) (708). In this fashion, the sensor data acts as input to the control algorithm that may drive decisions regarding drug delivery to the user.

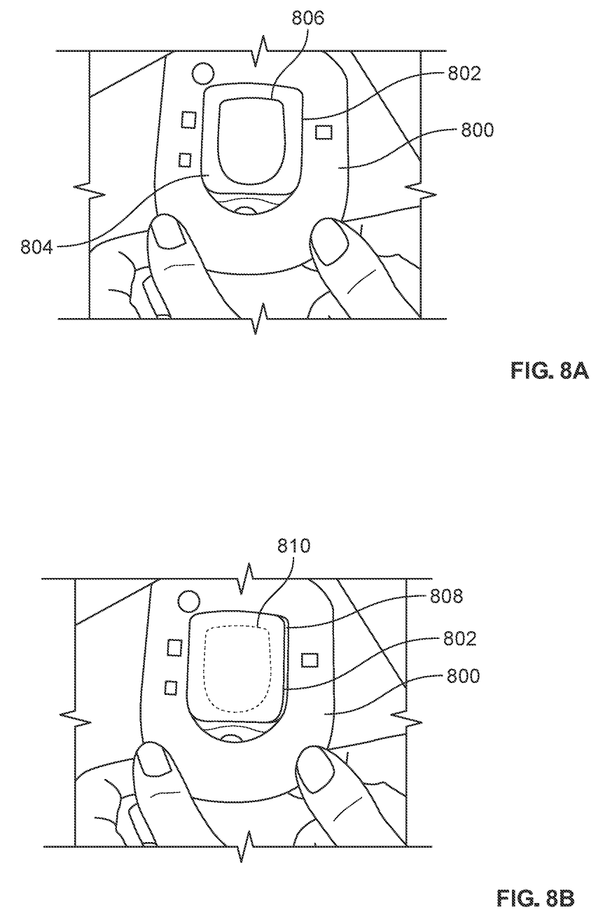

The metallic coil (404) adhesive pad (400) may engage the metallic coil of the drug delivery device (402) in different ways. FIG. 8A depicts a first way of engagement. In the approach of FIG. 8A, the adhesive pad (800) is secured over the drug delivery device (802). The metallic coil (806) is positioned near or on the top cover of the drug delivery device to couple with the metallic coil (404) within or below the adhesive pad (400) (FIG. 4). The adhesive pad (800) adheres in part to the top portion of the drug delivery device (802). Alternatively, as shown in FIG. 8B, the metallic coil (404) of the adhesive pad (800) may couple with the metallic coil (810) in the drug delivery device (802) via the bottom cover (808) of the drug delivery device (802). The metallic coil (810) may be positioned near or on the bottom cover (808).

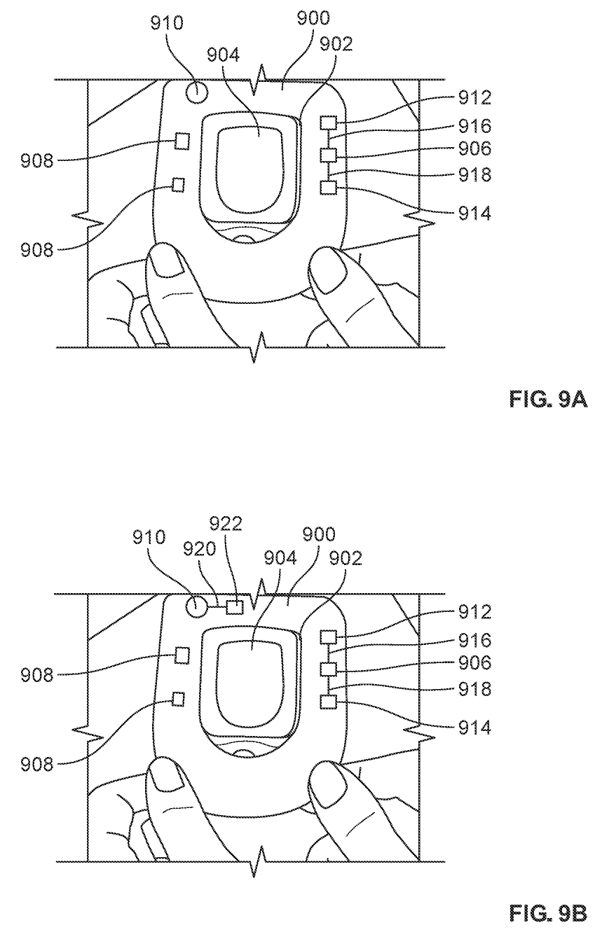

There may be wired connections between the drug delivery device (402) and the adhesive pad (400). In FIG. 9A, the adhesive pad (900) is positioned under the bottom cover (904) of the drug delivery device (902). The adhesive pad of the drug delivery device (902) is not visible but overlaps in an annular region like (210) in FIG. 2A. The adhesive pad may include a power source (910) and electronic components (906) and (908) that are secured to the adhesive pad (900). Alternatively, the electronic components (906) may be powered by the power source within the on-body drug delivery device. The adhesive pad of the drug delivery device (902) includes electronic components (912) and (914). In some exemplary embodiments, the electronic components (912) and (914) have respective electrical connections (916) and (918) with electronic component (906). The electrical connections (916) and (918) may be realized as conductive wires that extend between the adhesive pad (900) and the adhesive pad of the drug delivery device (902). The connections are not limited to between electronic components as shown in FIG. 9A. There also may be an electrical connection (920) between the power source (910) and an electronic component (922) on the adhesive pad of the drug delivery device (902) as shown if FIG. 9B. In such an arrangement, the power source (910) provides power to the electronic component (922) via the electrical connection (920). The depiction in FIG. 9B otherwise is like that of FIG. 9A.

While exemplary embodiments have been described herein, various changes in form and detail may be made relative to the exemplary embodiments that fall within the intended scope as defined in the appended claims.

1. A drug delivery system, comprising:
a drug delivery device for delivery a drug to a user, the drug delivery device including a processor configured for controlling delivery of doses of a drug to user by the drug delivery device
an adhesive pad for securing the drug delivery device to skin of the user, the adhesive pad having a geometry configured to at least partially surround the drug delivery device and an adhesive on one side of the adhesive pad for securing the adhesive pad to the skin of the user;
a sensor secured to the adhesive pad for sensing biometric information and generating output indicative of the sensed biometric information.

2. The drug delivery system of claim 1, further comprising a power source secured to the adhesive pad for providing power.

3. The drug delivery system of claim 2, wherein the power source is one of a battery power source or an energy harvesting device.

4. The drug delivery of claim 1, further comprising a wireless transceiver secured to the adhesive pad for facilitating wireless communications, including wireless communications between the sensor and the processor of the drug delivery device.

5. The drug delivery system of claim 1, wherein the sensor measures at least one of activity, heart rate, moisture, temperature, glucose, analytes, or sound pressure.

6. The drug delivery system of claim 1, further comprising a wired connection between the sensor and the drug delivery device.

7. The drug delivery system of claim 1, further comprising an additional sensor for sensing the biometric information of the user.

8. The drug delivery system of claim 1, wherein the adhesive pad only partially surrounds the drug delivery device.

9. The drug delivery system of claim 1, further comprising an additional adhesive pad for securing the drug delivery device to the skin of the user, wherein the adhesive pad at least partially overlaps the additional adhesive pad when secured to the skin of the user.

10. A drug delivery device, comprising:
a drug storage for storing a drug;
a pump for pumping the drug from the drug storage;
a cannula for delivering the drug subcutaneously to a user of the drug delivery device;
a processor configured to control delivery of drug doses to the user;
an adhesive pad for securing the drug delivery device to the skin of the user; and
a sensor secured to the adhesive pad for sensing biometric information of the user for use by the processor in controlling the delivery of the drug doses to the user.

11. The drug delivery device of claim 10, wherein the drug includes insulin.

12. The drug delivery device of claim 10, wherein the sensor measures at least one of activity, heart rate, moisture, temperature, glucose, analytes, or sound pressure.

13. The drug delivery device of claim 10, wherein the processor uses the biometric information sensed by the sensor to determine drug doses for delivery by the drug delivery device.

14. The drug delivery device of claim 10, further comprising a system on a chip (SoC) secured to the adhesive pad.

15. The drug delivery device of claim 10, further comprising a power source secured to the adhesive pad that powers the sensor.

16. A drug delivery system, comprising:
a storage media for storing computer program instructions;
a drug delivery device for delivering a drug to a user;
an adhesive pad for securing the drug delivery device to skin of the user;
a sensor secured to the adhesive pad for sensing biometric information of the user; and a processor configured to execute the computer programming instructions to cause the processor to:receive sensed biometric information from the sensor;use the sensed biometric information in determining a next drug delivery dose to be delivered by the drug delivery device.

17. The drug delivery system of claim 16, wherein the executing of the computer programming instructions by the processor further causes the processor to:
receive glucose level data of the user from a glucose sensor; and
use the glucose level data along with the sensed biometric information in determining the next drug delivery dose to be delivered by the drug delivery device.

18. The drug delivery system of claim 16, further comprising an additional sensor secured to the adhesive pad for sensing additional biometric information of the user.

19. The drug delivery system of claim 18, wherein the executing of the computer programming instructions by the processor further causes the processor to:
receive the additional biometric information of the user sensed by the additional sensor;
and use the glucose level data along with the sensed biometric information received from the sensor and the additional biometric information received from the additional sensor in determining the next drug delivery dose to be delivered by the drug delivery device.

20. The drug delivery system of claim 16, wherein the drug delivery device is a insulin patch pump.
